(19) United States
(12) Patent Application Publication
(10) Pub. No.: US 2008/0063720 A1
(43) Pub. Date: Mar. 13, 2008

(54) Delivery System

(76) Inventors: Iouri Kuzmich Gounko, County Dublin (IE); Yury Rakovich, Dublin (IE); Yuri Volkov, Dublin (IE); John Dongegan, County Kildare (IE); Dermot Kelleher, County Dublin (IE); Siobhan Mitchell, Dublin (IE)

Correspondence Address: JACOBSON HOLMAN PLLC, 400 SEVENTH STREET N.W., SUITE 600, WASHINGTON, DC, 20004, US

(21) Appl. No.: 11/587,775

(22) PCT Filed: Apr. 29, 2005
(86) PCT No.: PCT/IE05/00047

§ 371 Date: Oct. 27, 2006

Related U.S. Application Data
(60) Provisional application No. 60/566,085, filed on Apr. 29, 2004.

(51) Int. Cl. A61K 9/14; A61K 47/00; A61P 25/00

(52) U.S. Cl. 424/489; 514/772

(57) ABSTRACT

Therapeutic drug delivery and diagnostics systems comprise biologically active compounds associated with particulate carriers of less than 20 nm. These systems can be utilised for targeted modification of growth, development and functions, such as gene expression, protein synthesis, intracellular energy production and transport mechanisms in prokaryotic and eukaryotic organisms. The systems are also applicable for controlled modification of structural and functional properties of extracellular components and tissue constituents. The characteristics of a biological site are evaluated and an entity is provided which is dependent on the site characteristics. The entity comprises nanoparticles of less than 20 nm. A probe comprising nanoparticles of less than 5 nm is also provided.

### BACKGROUND

Traditional methods for delivery of biological compounds in vivo and in vitro rely on the use of soluble molecular substances or liposome-assisted transmembrane transport. However, the use of many biologically active compounds is limited due to their ubiquitous distribution and accumulation in various cells, or multiple tissue locations precluding specific accumulation of the compounds in selective target locations.

Recent interdisciplinary technological developments have led scientists to embrace nanoparticle methodology for biomedical applications (Bruchez et al., 1998; Chan et al., 1998; Akerman et al., 2002). Of a wide variety of nanoparticles available, quantum dots (QDs) in particular, or colloidal semiconductor nanocrystals are robust particles of size and composition tunable emission. They exhibit wide absorption profiles allowing excitation of various QDs simultaneously, narrow emission spectra and excellent photo stability (Mattoussi et al., 2002; Michalet et al., 2001; Chan et al., 2002), making them potentially readily traceable in the cells and tissues of the living organisms.

Initial hurdles of biocompatibility, solvent-based production, complex surface chemistry and low quantum yield have now been overcome allowing investigation of nanoparticle activity in biological systems (Chan et al., 1998; Bruchez et al., 1998). These advances include capping CdSe particles with ZnS to allow for an increased quantum yield (Chan et al., 1998), while Peng and colleagues have utilised alternative precursor materials to generate large quantities of high quality nanocrystals (Peng et al., 2001).

QDs display dimensional similarities to biomolecules permitting their bioconjugation and use as sensors. To date QD studies have been performed primarily using CdSe particles. Early attempts at labelling cells included adding transferrin-QD bioconjugates to HeLa cells thereby allowing receptor-mediated endocytosis (Chan et al., 1998). Also, the avidin-biotin system was employed to label F actin filaments where biotinylated CdSe nanocrystals were used to label fibroblasts incubated in phalloidin-biotin and streptavidin (Bruchez et al., 1998). CdSe—CdS nanocrystals coated with trimethoxysilylpropyl urea and acetate were found to bind with high affinity in the cell nucleus (Bruchez et al., 1998). CdSe QDs have also been used in metastatic assessment as markers for phagokinetic tracks (Parak et al., 2002). The first reports of in vivo use show QD-peptide conjugates targeting tumor vasculature (Akerman et al., 2002). Later studies using ZnS coated CdSe QDs encapsulated in PEG micelles show DNA binding and successful microinjection into Xenopus embryos (Dubertret et al., 2002).

Detection and selective functional modification of complex cell surface receptor repertoire, intracellular components and individual biomolecules in cell systems and in vitro applications constitute a priority task in modern biology and medicine. The most typical examples are drug screening, flow cytometry, cell imaging, protein and DNA detection. Traditional methods for detecting biological compounds in vivo and in vitro rely mostly on the use of radioactive markers. For example, these methods commonly use radioactive-labelled probes such as nucleic acids labelled with ³²P or ³⁵S and proteins labelled with ³⁵S or ¹²⁵I to detect biological molecules. These labels are effective because of the high degree of sensitivity for the detection of radioactivity. However, many basic difficulties exist with the use of radioisotopes. Such problems include the need for specially trained personnel, general safety issues when working with radioactivity, inherently short half-lives with many commonly used isotopes, and disposal problems due to full landfills and governmental regulations. As a result, current efforts have shifted to utilising non-radioactive methods of detecting biological compounds. These methods often consist of the use of fluorescent molecules as tags (e.g. fluorescein, ethidium, methyl coumarin, rhodamine, etc.), or the use of chemiluminescence as a method of detection. Fluorescence is the emission of light resulting from the absorption of radiation at one wavelength (excitation) followed by nearly immediate re-radiation usually at a different wavelength (emission). Fluorescent dyes are frequently used as tags in biological systems. For example, compounds such as ethidium bromide, propidium iodide, Hoechst dyes (e.g. benzoxanthene yellow) interact with DNA and fluoresce to visualize DNA. Other biological components can be visualized by fluorescence using such techniques as immunofluorescent microscopy, which utilizes antibodies labelled with a fluorescent tag and recognizing particular cellular target. For example, in a commonly used two-step immunodetection method, “secondary” polyclonal (rabbit- or goat-anti-mouse) antibodies tagged with fluorescein or rhodamine enable one to visualize “primary” monoclonal antibodies (typically raised in mice or respective hybridoma cells) bound to specific cellular targets. However, simultaneous use of several “primary” murine monoclonal antibodies to detect multiple targets is limited by the species specificity of the “secondary” fluorescently-tagged reagents leading in this case to severe cross-reactivity and false positive staining results. In one aspect the invention is directed to providing a solution to this problem.

Fluorescent dyes also have applications in non-cellular biological systems. For example, the advent of fluorescently-labelled nucleotides has facilitated the development of new methods of high-throughput DNA sequencing and DNA fragment analysis (ABI system; Perkin-Elmer, Norwalk, Conn.). Despite certain progress, there are a number of chemical and physical limitations to the use of organic fluorescent dyes. One of these limitations is the variation of excitation wavelengths of different coloured dyes. As a result, simultaneously using two or more fluorescent tags with different excitation wavelengths requires multiple excitation light sources. This requirement thus adds to the cost and complexity of methods utilising multiple fluorescent dyes. Another drawback when using organic dyes is the deterioration of fluorescence intensity upon prolonged exposure to excitation light. This fading is called photobleaching and is dependent on the intensity of the excitation light and the duration of the illumination. In addition, conversion of the dye into a nonfluorescent species is irreversible. Furthermore, the degradation products of dyes are organic compounds, which may interfere with biological processes being examined. Another drawback of organic dyes is the spectral overlap that exists from one dye to another. This is due in part to the relatively wide emission spectra of organic dyes and the overlap of their spectra near the low energy region. Few low molecular weight dyes have a combination of a large Stokes shift, which is defined as the separation of the absorption and emission maxima, and high fluorescence output. In addition, low molecular weight dyes may be impractical for some applications because they do not provide a strong enough fluorescent signal. Furthermore, the differences in the chemical properties of standard organic fluorescent dyes make multiple, parallel assays quite impractical since different chemical reactions may be involved for each dye used in the variety of applications of fluorescent labels.

Practical aspects of bioconjugation of thiol-stabilized CdTe nanoparticles with complementary antigen and antibody have been reported in the literature (Wand et al, 2002). However the bioactivity of the prepared immunocomplexes in this case was limited. Moreover, the size of nanoparticles was not precisely controlled. The possibility of the lymph node mapping was demonstrated by Kim et al (2004) using CdTe/CdSe core-shell nanocrystals. However, the use of these nanocrystals is restricted to applications where there is not significant absorption of infrared emission by biological tissue. An additional problem is the toxicity of such a composite, which limits the possible applications. The use of CdSe/ZnS nanocrystals as fluorescent labels for multiphoton microscopy was recently demonstrated by Larson et al (2003). Although the authors visualized quantum dots dynamically through the skin of living mice, this method is of limited usefulness because high pumping intensity is a critical requirement to achieve efficient multiphonon assisted excitation of nanocrystal luminescence. A direct method for conjugating protein molecules to luminescent CdSe—ZnS core-shell nanocrystals was described by Mattoussi et al (2000) and later by Goldman et al (2002). These bioconjugates have been proposed as bioactive fluorescent probes in sensing, imaging, immunoassay and other diagnostic applications. However, the bioconjugates are of relatively large size (30-45 nm in diameter) and had a quite limited solubility in water. As result these nanocomposites have only limited capability to penetrate through the cell membrane and can not be used very effectively for intracellular diagnostics. Also, water-soluble CdTe, CdₓHg₍₁₋ₓ₎Te and HgTe nanocrystals have been proposed for biolabeling of biocompatible polymers. In this work the nanocrystals were encapsulated into the polymer with the formation of microcapsules, which have been suggested as potential materials for monitoring the drug delivery process (Gaponik et al, 2003). Although the initial CdTe or HgTe nanocrystals demonstrated good water solubility and were of small size (4-6 nm) the final composites with the biopolymer were of several micron sizes and were too large to be used for intracellular drug delivery and diagnostics.

The invention is directed towards solving at least some of the problems with known systems.

### SUMMARY OF THE INVENTION

According to the invention there is provided a method for targeting a specific site comprising the steps of: —
evaluating characteristics of a biological target site; and
providing an entity dependent on the site characteristics, the entity comprising nanoparticles of up to 20 nm to target the entity to the specific site.

The target site may be evaluated by in vivo or in vitro means. Typically in vitro techniques may be executed, which may include, extraction of cells, phenotypic or genotypic examination of their function by light and ultra microscopic analysis, fluorescent microscopy, computer assisted 3 D reconstructions, biochemical analysis, proteomics and mathematical prediction and modelling and the like.

In one embodiment the entity comprises nanoparticles of up to 10 nm. In one case the entity comprises nanoparticles of up to 5 nm. In one embodiment the entity comprises nanoparticles of up to 3 nm.

The nanoparticles may all be of the same type or there may be a mixture or combination of different nanoparticles.

The nanoparticles may be water soluble and/or lipid soluble.

In one embodiment the nanoparticles comprises II-VI nanoparticles such as CdTe nanoparticles or CdSe nanoparticles.

In one embodiment the method comprises modifying the material composition, charge, or any surface parameters of the entity.

In one case the target is living cells. The target may be dead cells. The target may be a bacterium, a fungus, a eukaryotic life form, a prokaryotic life form. The target may be intracellular organelles. The target may be within or on a biological membrane. The target may be extracellular to a biological membrane. The target may be mitochondria. The target may be endoplasmic reticulum.

In one embodiment the target are cells of the immune system. The cells of the immune system may be lymphocytes T and B cells, neutrophils, eosinophils, basophils, monocytes, macrophages, dendritic cells and other antigen presenting cells, precursor cells and cells performing immune functions in other tissues such as astrocytes, glial cells and neurons and undifferentiated cells such as stem cells.

The target may be macrophages. The target may be the nucleus of macrophages. The target may be the nucleolus of phagocytes. The target may be the cytosol of macrophages.

In one embodiment the target is a cellular or an acellular component of the blood coagulation system. The target may be platelets, neutrophils, and/or fibrin.

In one embodiment the entity is directly associated with a nanoparticle. The entity may be directly linked to a nanoparticle. The entity may be physically attached to a nanoparticle. The entity may be chemically attached to a nanoparticle.

The entity may be conjugated to a nanoparticle.

In another embodiment the entity is indirectly associated with a nanoparticle. The entity may be indirectly linked to a nanoparticle using an organic linker.

The entity may comprise a stabiliser.

In one embodiment the entity comprises a medicinal drug. Such systems comprising nanoparticles linked to medicinal drugs may be selectively targeted to enhanced transport across cellular and subcellular membranes and blood organ barriers as well as selectively cut off from entering the site due to size, charge, shape and other characteristics.

In another embodiment the entity comprises a diagnostic or imaging agent or sensor. The entity may comprise DNA. The entity may comprise DNA, RNA, protein, or chemical, their derivatives, constituents thereof and modified products thereof.

In another aspect the invention provides a drug delivery system comprising a biologically active entity associated with a carrier comprising nanoparticles of up to 20 nm.

In one case the entity is directly associated with a nanoparticle. The entity may be directly linked to a nanoparticle. The entity may be physically attached to a nanoparticle. The entity may be chemically attached to a nanoparticle. The entity may be conjugated to a nanoparticle.

In another case the entity is indirectly associated with a nanoparticle. The entity may be indirectly linked to a nanoparticle using an organic linker.

The entity may comprise a stabiliser.

In one embodiment the carrier comprises nanoparticles of up to 10 nm. The carrier may comprise nanoparticles of up to 5 nm. The carrier may comprise nanoparticles of up to 3 nm.

The nanoparticles may all be of the same type or there may be a mixture or combination of different nanoparticles. The nanoparticles may be water-soluble and/or lipid soluble.

In one embodiment the nanoparticles comprise water-soluble II-VI nanoparticles such as CdTe nanoparticles or CdSe nanoparticles.

The system may be used for targeting a biological object such as living cells, dead cells, a bacterium, a fungus, a eukaryotic life form, a prokaryotic life form, intracellular organelles.

The target may be within or on a biological membrane. The target may be extracellular to a biological membrane.

The target may be mitochondria, endoplasmic reticulum, cells of the immune system, macrophages, the nucleus of macrophages, the nucleolus of phagocytes, or the cytosol of macrophages.

In one embodiment the target is a cellular or an acellular component of the blood coagulation system. The target is platelets, neutrophils, or fibrin.

The nanoparticles may be II-VI nanoparticles.

The nanoparticles may be organic or inorganic nanoparticles.

The entity may interact with and undergo modifications upon contact with cytoplasmic components, and in turn modify their function.

The entity may interact with and undergo modifications upon contact with cytoskeletal components, and in turn modify their function.

The entity may interact with and undergo modifications upon contact with nuclear components, and in turn modify their function.

The entity may interact with and undergo modifications upon interaction with extracellular matrix, and in turn modify their function.

The entity may interact with and undergo modification with blood constituents.

The entity may interact with and undergo modifications upon interaction with cell membrane, membranes of subcellular organelles, nuclear membrane and/or nuclear pores.

The entity may be targeted to selected intracellular compartments by an external magnetic field.

The entity may be targeted to selected intracellular compartments by external optical illumination.

The entity may be targeted to selected intracellular compartments by modification of intracellular pH.

The entity may be targeted to selected subcellular organelles by external magnetic field.

The entity may be for treatment of cancer and other diseases accompanied by abnormal cell and tissue function. The entity may be for treatment of inflammatory conditions.

The entity may be targeted to the intestinal epithelium, bacterial or parasitic flora utilised for treatment of infections of gastrointestinal tract.

The entity may be for treatment of coagulation disorders and cardiovascular diseases.

In another aspect the invention provides a probe comprising nanoparticles of less than 5 nm in size. The invention also provides a probe comprising nanoparticles of less than 3 nm in size. The nanoparticles may be of CdTe.

The probe may be targeted to cellular or extracellular components and may be used for example in the treatment of blood coagulation disorders associated with excessive clot formation and thrombosis.

According to a further aspect the invention provides a drug delivery system, especially a drug delivery system comprising a biologically active compound chemically or physically linked to a particulate carrier of nanometric size for controlled delivery of the compound into a target.

In one embodiment the system is water soluble.

The target may be a living cell. The target may be a subcellular organelle or compartment. In another case the target is a cell nucleus. The target may be a component of the extracellular matrix.

In one embodiment the target is a component of the blood coagulation system. In a preferred embodiment the carrier is a nanoparticle.

The carrier may be a water-soluble II-VI colloidal nanoparticle.

In one case the carrier exhibits photoluminescence having a quantum yield of at least 1% in water. The carrier may exhibit photoluminescence having a quantum yield of at least 10% in water.

In one embodiment the carrier comprises a core from a II-VI semiconductor and an organic stabiliser with different functionalities such as carboxylic acids, amines, alcohols, aldehydes, esters, peptides, their derivatives or any other functional groups.

The carrier may comprise water-soluble magnetic nanoparticles.

In another embodiment the carrier comprises organic and inorganic (e.g. polyhedral silsesquioxanes) polymer nanoparticles.

The compound may interact with and undergo modifications upon contact with cytoplasmic components, cytoskeletal components, nuclear components, extracellular matrix, liquid blood constituents, cell membrane, membranes of subcellular organelles, nuclear membrane and/or nuclear pores.

The carrier may be targeted to selected intracellular compartments by an external magnetic field, external optical illumination or by modification of intracellular pH.

The carrier may be targeted to selected subcellular organelles by external magnetic field.

The biologically active compound may be useful for treatment of cancer and other diseases accompanied by abnormal tissue proliferation.

In one case the biologically active compound is useful for treatment of inflammatory conditions. Inflammation is the body's response to injury, infection or molecules perceived by the immune system as foreign. Although the ability to mount an inflammatory response is essential for survival, the ability to control inflammation is also necessary for health. Absent, excessive or uncontrolled inflammation results in a vast array of diseases that includes the highly prevalent conditions of allergy, asthma, arthritis, autoimmune conditions, including systemic lupus erythematosus, dermatomyositis, polymyositis, inflammatory neuropathies (Guillain Barré, inflammatory polyneuropathies), vasculitis (Wegener's granulomatosus, polyarteritis nodosa), and rare disorders such as polymyalgia rheumatica, temporal arteritis, Sjogren's syndrome, Bechet's disease, Churg-Strauss syndrome, and Takayasu's arteritis, inflammatory muscle disorders, inflammatory bowel disease, psoriasis, and multiple sclerosis, kidney (glomerular) and liver disease, Chronic obstructive pulmonary disease (COPD), Cardiovascular disease, inflammatory disease of the CNS e.g. bacterial meningitis and encephalitis. Also of interest is cardiovascular disease, blood disorders, clotting disorders, AIDS, TB, malaria, hemopoietic malignancies (leukaemia) and neutropenia, cancers originating from epithelial and non epithelial origin, hemophilia, stroke, gastrointestinal inflammation, neuroinflammatory disorders and transplantation.

Another embodiment of the invention is the use of nanosized drug delivery systems for treatment of diseases caused or associated with abnormal, increased or decreased blood coagulation such as bleeding and blood loss of traumatic and non traumatic origin, haemophilia, non inherited blood disorders, deficiencies of individual components of blood coagulation cascade, or diseases related to excessive blood clot formation thrombosis, stroke, heart attack and ischemic condition with different organ and tissue localisation. For example nanosized drug delivery systems may represent anti coagulant drug linked to the nanoparticles with ability to selective influence the formation of blood clot components such as fibrin but not limited to. Alternatively without limitation to this example nanosized drug delivery systems can represent nanoparticles linked to but not limited to Factor VIIa and used to reduce the clotting time, whether applied topically or systemically.

The biologically active compound may be targeted to the intestinal epithelium, bacterial or parasitic flora utilized for treatment of infections of gastrointestinal tract.

In one case the biologically active compound is useful for treatment of coagulation disorders and cardiovascular diseases.

The invention provides drug delivery, diagnostics and molecular visualisation systems based on structures consisting of biologically active compounds coupled to nanoscale-size carriers, hereinafter referred to as “nanodrug systems”.

The invention provides new drug delivery systems, which are significantly (an order of magnitude) smaller than previously reported systems, being based on nano-size particle carriers (nanodrug systems). The drug delivery systems comprise biologically active compounds linked to particular carriers of nanometric size suitable for targeting the compounds, for example into living cells.

The new systems enable selective targeting of the biologically active compounds or medicinal drugs to the cells and intracellular compartments based on the size, charge and/or chemical properties of the carriers.

The systems can be selectively targeted to selective organs, tissues, cells and subcellular organelles on the basis of size, charge and surface chemistry characteristics of the particular carrier.

In specific embodiments, the invention offers the possibility of controlled custom modification of chemical properties of the nanodrug systems.

It is envisaged that the efficiency of drug delivery utilising the nano systems can be directly traced and monitored in the organs, tissues and individual cells.

Drug delivery can be selectively regulated by external application of magnetic fields (for nanodrug composites based on magnetic carriers), optical illumination and/or modification of pH in the physiologically buffered systems.

The invention provides a system for reliably performing monoclonal antibodies directly labelled with fluorescent compounds, possessing unique and clearly distinguishable colour emission characteristics using a range of nanocrystals. In addition, the need for “secondary” polyclonal reagents is eliminated thus significantly reducing the costs of the method and last, but not least contributing to the establishment of animal-free experimental systems in biomedical practice.

The principal differences between the nanodrug systems of the present invention and existing or reported potentially exploitable drug delivery techniques are the following, not excluding other possible significant advantages:

Conventional oral, intranasal, parenteral, intravenous and intra-peritoneal drug delivery systems are essentially based on passive distribution of water- or lipid-soluble substances within the organism, hence lacking the overall selectivity of drug penetration into the living cells and thereby frequently causing undesirable side effects. The same applies to the conventional in vitro systems where biologically active compounds are brought in contact with the cells by simple direct addition (mixing) into the culture media. In contrast, the delivery systems of the invention can be selectively targeted to selective organs, tissues, cells and subcellular organelles on the basis of size, charge and surface chemistry characteristics of the particular carrier. The counteracting physiological barriers (bio-filters) determining the accessibility of the nanodrug systems to the targets could be imposed by cell and subcellular organelle (including nucleus) membranes and intracellular compartments. These may also include intercellular gap junctions, blood-brain, placental and other physiological barriers at the cell, tissue and organ level.

Other more recently reported drug delivery systems representing complexes or enclosed compartments of drugs and microcarriers although permitting improved selectivity of the compounds delivery, are nonetheless intrinsically limited by the micrometric scale size of the carriers. Hence certain tissue and/or cellular structures with the size parameters below predetermined limits are excluded as potential targets. These limitations do not apply to the nanodrug systems of the invention as they are of an order of magnitude smaller compared to the currently available analogs.

The suggested biomedical research applications of nanoparticles in living cells have so far been mostly confined to cell membrane proteins detection utilising fluorescent quantum dots. These studies have been performed on relatively large core-shell particles (commonly over 16 nm in diameter) significantly limiting bio-accessibility of the used systems, as their size was approaching the dimensions of the antibodies and other commonly expressed and secreted proteins thereby potentially masking or inhibiting important molecular interactions. The systems of the invention overcome these limitations as they are based on a carrier, and do not employ the core-shell structure of the nanoparticle.

Due to the difficulties with intracellular delivery of quantum dots, previously published studies were limited to applications dealing with proteins and receptors expressed on the outer surface of the cell membrane and did not involve studies at the subcellular level since the detection systems were too large to be cell permeable (Bruchez et al, 1998, Chan et al, 2002). The delivery systems of the invention have the substantial and crucial advantage of being capable of rapid accumulation inside the cells by means of phagocytosis, pinocytosis, endocytosis or/and cytoskeletal, organelle and other particle transport mechanisms. In addition selective accumulation of nanosized drug delivery systems within the target site can be influenced, facilitated or inhibited by physical, chemical or other processes including but not limited to diffusion, enhanced diffusion, electroporation and other transfection procedures, gradients, semi-permeability and direct injection into organs, cells and tissues.

Other studies demonstrating targeted labelling of intracellular structures or proteins using quantum nanodots were performed on fixed cells subsequently detergent extracted (permeabilised) to enable detection system intracellular accessibility. This approach a priori incorporates artefacts imposed by the cell fixation in comparison to the experiments in the living cells. The nanodrug systems of the present invention are suitable for applications in the living cells thereby avoiding such fixation artefacts. We were able to study intracellular distribution dynamics of nanodrug systems in living cells over considerable periods of time.

### BRIEF DESCRIPTION OF THE FIGURES

The invention will be more clearly understood from the following description thereof given by way of example only with reference to the accompanying figures in which: —

FIG. 1 are images acquired by fluorescent microscopy illustrating an intracellular distribution of red-emitting quantum dots (QDs) in human primary macrophages (upper and middle panel) and corresponding phase contrast image (lower panel);.

FIG. 2 are images acquired by fluorescent microscopy illustrating a phase contrast (upper) and confocal (lower) images of CdTe nanocrystals with two distinctive fluorescence spectra microinjected into cultured transformed epithelial cell line HT29;

FIG. 3 are images acquired by fluorescent microscopy illustrating a selective intracellular distribution of red (left panel) versus green (right panel) quantum dots in the living phagocytic cells visualized by confocal microscopy. Left panel, thick white arrow highlights the endoplasmic reticulum and thin white arrow shows the nucleolus;

FIG. 4 are images acquired by fluorescent microscopy illustrating specificity of intranuclear accumulation of green-emitting QDs in human primary macrophages. After simultaneous intracytoplasmic injection of green and red-emitting QDs only the green particles display a characteristic nuclear pattern (lower panel thick white arrow). Red QDs are present in the cytosol and in the discrete perinuclear location (rough Endoplasmic reticulum) (upper panel, thick white arrow);

FIG. 5 is an image acquired by fluorescent microscopy showing a thapsigargin-induced blockade of intranuclear accumulation of quantum dots in human primary macrophage cells;

FIG. 6 is an image acquired by fluorescent microscopy showing a brefeldin A-induced dispersion of quantum dots and partial block of intranuclear accumulation in macrophages;

FIG. 7 are images acquired by fluorescent microscopy illustrating an accumulation of 2.2 nm size green-emitting quantum dots in freshly formed fibrin filaments. Left panel, fluorescence in the green channel. Right panel, corresponding microscopic field in transmitted light showing two large polymorphonuclear cells (neutrophils) and six red blood cells;

FIG. 8 are images acquired by fluorescent microscopy illustrating an accumulation of aspirin—functionalised siloxane nanoparticles in normal peripheral blood polymorphonuclear cell (neutrophil). Cells were incubated in the presence of the nanoparticles for 30 mins and subsequently analysed by live cell confocal microscopy. A, upper optical plane (top of the cell), B, middle plane showing highlighted segmented nucleus of the polymorphonuclear cell (arrow), due to accumulation of fluorescent drug-coupled nanoparticles; C, lower optical plane (at the level of cell contact with glass support);

FIG. 9 is an image acquired by fluorescent microscopy illustrating the accumulation of red CdTe nanoparticles in the mitochondria of macrophages. Cells were incubated with nanoparticles for 15 mins at 37° C. and subsequently analysed by live cell confocal microscopy Arrowhead points to the nucleus of the cell (free from nanoparticles). Arrows indicate individual mitochondria in the cytoplasm; and

FIG. 10 are images acquired by fluorescent microscopy illustrating the accumulation of red and green CdTe nanoparticles in the blood coagulation system. Upper panel shows red channel with nanoparticles highlighting groups of platelet attached to the bottom of the culture well. Arrows show groups of platelets. Middle panel corresponds to the green channel depicting a meshwork of fibrin filaments decorated by green emitting nanoparticles. Arrow shows fibrin meshwork. Bottom panel combination panel shows overlayed red and green and blue channel. Nuclei of the cells were stained with blue emitting Hoechest stain. Thin arrows show cell nuclei. Fresh blood was incubated with nanoparticles for 15 mins at 37° C., until the formation of the clot was complete and subsequently analysed by live cell confocal microscopy.

### DETAILED DESCRIPTION OF THE INVENTION

### Definitions

Living cell (Cell)—refers to the self-replicating biological structure enclosed by an outer membrane and containing cytoplasm, organelles and nucleic acids (i.e. viruses, prokaryotic bacterial cells, protozoa and eukaryotic cells of higher species and multicellular organisms).

Carrier—rigid physical structure with nanosized core ranging between 1-100 nm.

Drugs—any chemical substances of therapeutic and/or diagnostic application. Nanoparticles are nanosized (between 1.0 and 100 nm) inorganic or organic particles with size dependent physical properties. These may include metal semiconductor, magnetic, organic or inorganic (e.g. polyhedral silsesquioxane) polymer nanoparticles.

Extracellular matrix—refers to the amorphous and fibrillar components of tissues and blood including collagen, laminin, fibronectin, vitronectin, their subtypes and combinations and other components thereof.

Coagulation components—refers to the entire plurality of factors participating in the process of blood clot formation, whether in soluble or fibrillar form.

Biologically active compounds—substances which are able to interact with the cells, biological membranes, subcellular components or nuclei and/or are capable of affecting cell or organelle function, proliferation or development as a result of such interactions.

II-VI colloidal quantum dots—are semiconductor nanoparticles of II-VI compounds prepared as a colloidal solution with size-dependent optical and electronic properties.

Optical illuminators/emitters—any source of ultraviolet, visible or infrared light and combinations thereof.

Chemical or physical linking—bond via covalent, noncovalent, hydrophobic, hydrophilic, electrostatic, van der Waals, hydrogen bonding, magnetic or electromagnetic interactions.

Cytoplasmic and nuclear components—refers to the plurality of proteins and protein derivatives (glycoproteins, nucleoproteins and other complex protein derivatives), nucleic acids (DNA, RNA), carbohydrates, lipids, glycolipids and other molecular cell constituents.

Synthesis of CdTe Nanoparticles

CdTe nanocrystals capped with thioglycolic acid used in the experiments were synthesized in aqueous medium as reported earlier (Gaponik et al, 2002). Briefly, demineralised aqueous solutions containing Cd(ClO₄)₂.6H₂O and a stabilizer (thioglycolic acid, TGA) at pH 11.8 were treated by H₂Te gas, which was generated by the reaction of Al₂Te₃ lumps with 0.5 M H₂SO₄ under nitrogen. The mixture of was then heated under reflux under open-air conditions. This method enabled us to prepare good quality CdTe nanocrystals with a narrow (<10%) size distribution. Variation of the temperature and the duration of the heating during the preparation of CdTe nanocrystals determines the final size of the nanocrystals and as a result the colour and luminescence maximum of the solution. Thus green (with photoluminescence maximum at 563 nm) CdTe nanoparticles were produced after 15 min of heating under reflux, while red (with photoluminescence maximum at 602 nm) CdTe colloid solution were produced after 24 hours of heating.

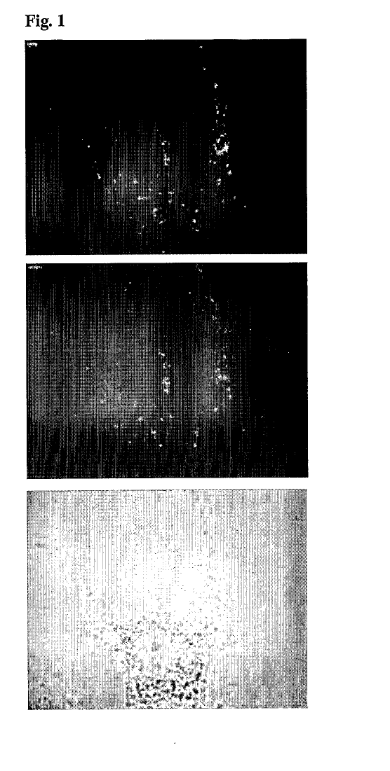

We have utilised water-soluble thioglycolic capped CdTe nanoparticles of varying sizes for selective nuclear and nucleolar localisation of green CdTe QDs and cytoplasmic compartmentalisation of red QDs, dependent on size and surface chemistry. CdTe nanoparticles showed limited cytotoxicity and proved to be suitable for biological systems as demonstrated by FIGS. 1 to 10.

The entity may comprise a stabiliser such as any thiol based organic stabiliser with different functionalities such as carboxylic acids, amines, alcohols, aldehydes, esters, amides, phosphines, alkyl-phosphates, their derivatives or any other functional groups. Table 1 lists examples of stabilizers, which may be used particularly with CdTe nanoparticles.

TABLE 1

| | PL quantum yield | Additional |
|---|---|---|
| | of as-prepared | commentson |
| Stabilizers | CdTe QDs | the stabilizer |
| 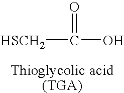 | 17% | A useful chemical intermediate in the chemical reactions such as addition, elimination and cyclization |
| 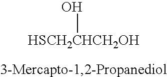 | 12% | It can be possibly used for bio-conjugation via ester bond |
| 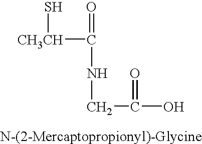 | 4%-6% | This product is used as chemotherapy and chemotherapy protection agent, liver protection agent and heavy metal antidote |
| 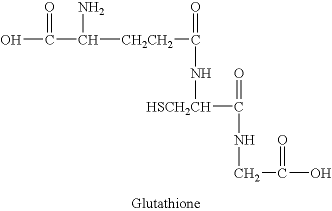 | 25%-30% | Glutathione has a facile electron-donating capacity, linked to it's sulfhydryl(-SH) group. Biologically, it is an important water- phase antioxidant and essential cofactor for antioxidant enzymes, it also provides protection for mitochondria against endogenous oxygen radicals |
| 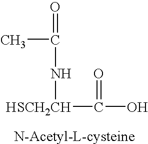 | 23% | Cysteine is important biologically for homeostasis, being a key antioxidant, a glutathione precursor, and a natural source of sulfur for metabolism. N-Acetyl Cysteine, is more stable than L-cysteine and conveniently becomes converted into L-cysteine after being absorbed. |
| 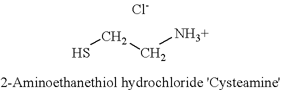 | 2% | Antioxidant protecting agent, prevent chemical changes caused by exposure to oxygen. Often displays chelating properties, masking heavy metal ions in solution. |

Cell and Tissue Culture Experiments

In the studies referred to in FIGS. 1-10, Human THP-1 monocytes, the transformed epithelial cell lines HT-29, HCT-116 and T cell lymphoma cell line HUT-78 were obtained from the European Collection of Animal Cell Cultures (ECACC, Salisbury, UK). Cells were grown in RPMI 1640 medium supplemented with 10% heat-inactivated fetal bovine serum (FBS), 2 mM L-glutamine/L, 100 μg penicillin/ml and 100 mg streptomycin/ml, and incubated at 37° C. in 5% CO₂. To induce monocyte to macrophage differentiation, THP-1 cells were cultured in the presence of 100 ng/ml PMA for 72 h.

Live cell imaging was performed in Lab-Tek chambered coverglass slides (Nunc). Microinjection was carried on an inverted Nikon TE300 microscope with Narishige hydraulic micromanipulation and microinjection equipment and utilising ex tempore fabricated glass capillary microneedles.

Images were acquired by fluorescence microscopy (Nikon Eclipse TE 300) and on the UltraView Live Cell Imager confocal microscopy workstation (Perkin-Elmer Life Sciences, Warrington, UK) (Nikon Eclipse TE 2000-U). Processing and 3-D image analysis was performed using Ultra View LCI and Volocity-2 software.).

FIG. 1 illustrates intracellular distribution of red-emitting 4.4 nm thiol-capped CdTe quantum dots in human primary macrophages (upper and middle panel) and corresponding phase contrast image (lower panel). Primary human macrophages were derived from the peripheral blood of healthy volunteers by initial positive selection of monocytes from the mononuclear blood fraction by adhesion to the surface of borosilicate glass chambered coverslips and subsequent maturation over 7-14 days in the presence of complete tissue culture medium supplemented with 10% fetal calf serum and antibiotics. Quantum dots were added to the differentiated macrophages at the stage of established cell spreading and incubated in complete culture medium over 1-24 hour intervals. Macrophages in these conditions did not show signs of excessive cell death even after the longest incubation intervals used thereby indicating suitability of nanoparticles for this type of biological application and limited cytotoxicity. FIG. 1 shows fluorescent images of the macrophages following an 18 hour incubation in the presence of quantum dots taken with a 25 min interval (upper panel reflect starting point, lower panel—end point). A significant change in localization of quantum dots over time will be noted which reflects their active intracellular transport.

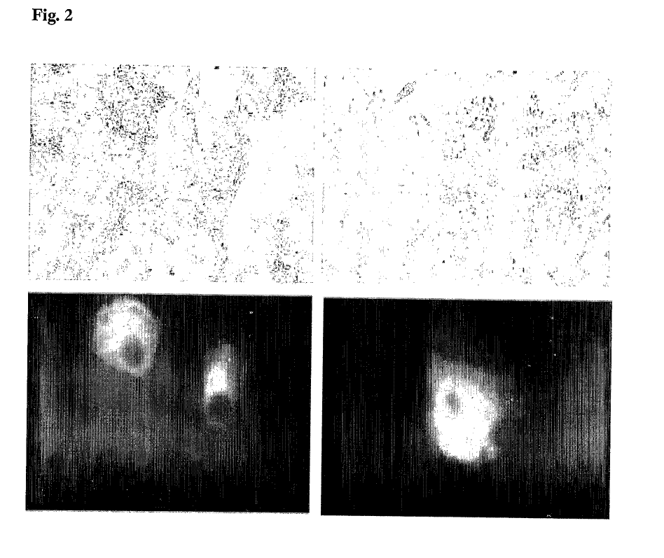

FIG. 2 illustrates phase contrast (upper) and confocal (lower) images of CdTe nanocrystals (green-emitting 2.2 nm and red-emitting 4.4 nm sizes, synthesised as described above) with two distinctive fluorescence spectra microinjected into cultured transformed epithelial cell line HT29. Human colonic epithelial carcinoma cells HT29 were split and seeded after the 4^{th} passage into the compartments of the 4-well borosilicate chambered coverslips and allowed to grow to a sub-confluent state. Nanoparticles were delivered into the cells via a direct intracytoplasmic injection with an ex tepmore pulled glass microinjection needle (inner diameter of the injecting tip 0.1-0.15 μm). Two types of quantum dots as described above with distinctive spectral properties were utilized in this experiment: lower left panel, red 4.4 nm-size quantum dots; lower right panel, green-emitting 2.2 nm size nanodots. Particles readily distribute inside the cells giving a bright fluorescent signal. Visualization, 100× oil immersion objective lens on a Nikon Eclipse TE 300 microscope with Leica DC-100 colour digital camera.

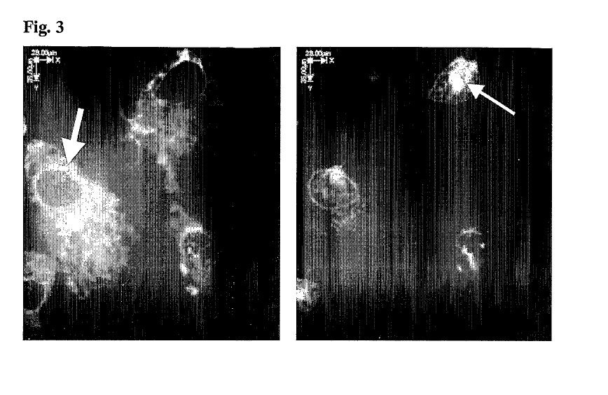

FIG. 3 illustrates selective intracellular distribution of green versus red quantum dots in the living phagocytic cells visualized by confocal microscopy. Green CdTe QDs localise in the nucleus of THP-1 cells while red CdTe QDs are concentrated in the cytoplasm. Differentiated THP-1 cells were washed three times with HBSS prior to their incubation with green or red CdTe particles. Particles were added in full media (2 μl green CdTe nanoparticles+2 μl red CdTe nanoparticles into 150 μl cell culture chamber). Cells were analysed after 30 mins. Right panel, fluorescence of red QDs detected in the red channel; left panel, fluorescence of green QDs detected in the green channel. Areas of pronounced quantum dots accumulation in close proximity to the nucleus correspond to the typical location of rough endoplasmic reticulum in mammalian cells.

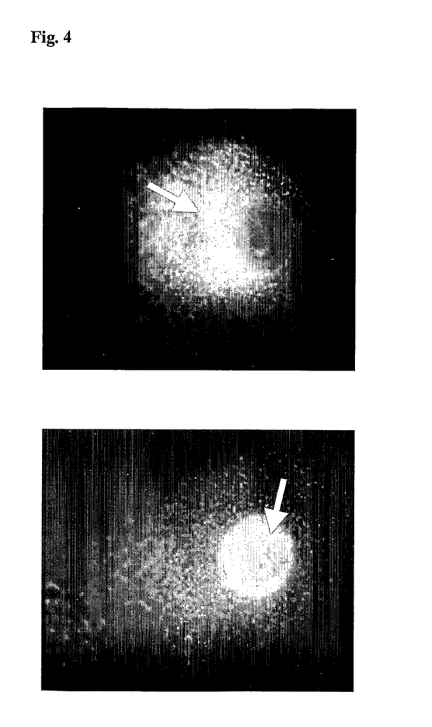

FIG. 4 illustrates red-emitting 4.4 nm particles do not undergo nuclear accumulation even if the primary barrier (cell membrane barrier) in macrophages is omitted by direct cytoplasmic microinjection of CdTe nanoparticles (FIG. 1) thereby suggesting the primary importance of active intracellular transport mechanisms, not necessary directly dependent on the phagocytic activity. Lower panel shows green-emitting 2.2 nm CdTe particles are retained in the nucleus following intranuclear microinjection. Nanoparticles were delivered into the cells via a direct injection with an ex tepmore pulled glass microinjection needle (inner diameter of the injecting tip 0.1-0.15 μm).

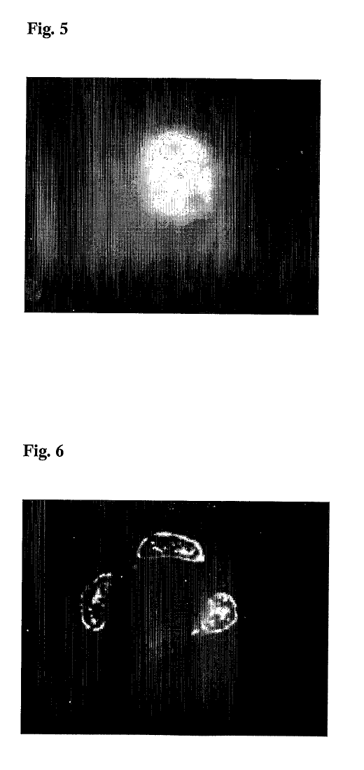

FIG. 5 illustrates thapsigargin-induced blockade of intranuclear accumulation of quantum dots in human primary macrophage cells. Nuclear import inhibitor thapsigargin (100 mM) was added to the cells for 30 min and subsequently washed out with pre-warmed culture medium prior to incubation with 2.2 nm size (green emitting) CdTe particles.

FIG. 6 illustrates brefeldin A-induced dispersion of quantum dots and partial block of intranuclear accumulation in macrophages. Prior to incubation with CdTe particles THP-1 differentiated macrophages were incubated with the Golgi complex disrupter Brefeldin A for 30 min at 20 μg/ml. Following a wash out, green 2.2 nm size CdTe particles were added to the cells.

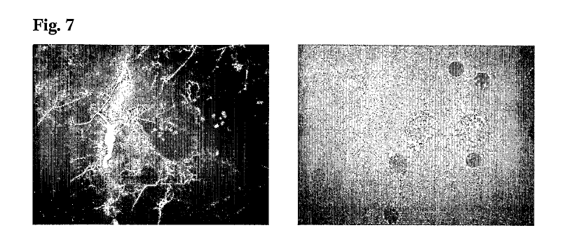

FIG. 7 illustrates accumulation of 2.2 nm size green-emitting quantum dots in freshly formed fibrin filaments. Left panel, fluorescence in the green channel. Right panel, corresponding microscopic field in transmitted light showing two large polymorphonuclear cells (neutrophils) and six red blood cells. Neutrophils were isolated from peripheral blood by adhesion onto the glass surface in chambered coverslips with subsequent washout of the unbound cells. Immediately after the washout, chambers were filled with fresh warm culture medium containing quantum dots and incubated at 37° C. for 30 minutes. Fibrin filaments start building up in these conditions after 5-10 min incubation period with neutrophils serving as primary sites initiating fibrin formation.

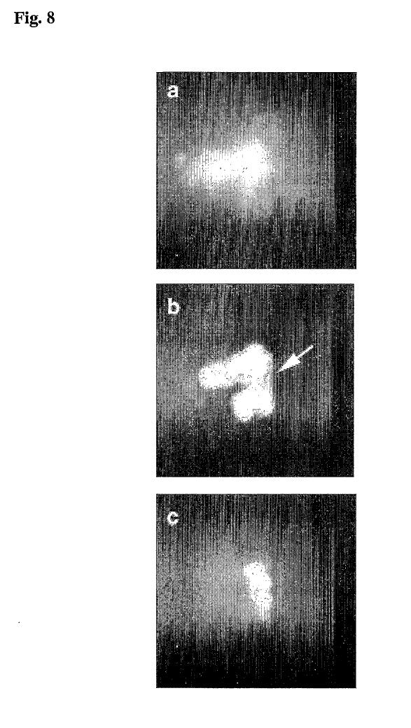

FIG. 8 illustrates accumulation of aspirin—functionalised siloxane nanoparticles in normal peripheral blood polymorphonuclear cell (neutrophil). Cells were incubated in the presence of the nanoparticles for 30 mins and subsequently analysed by live cell confocal microscopy. A, upper optical plane (top of the cell), B, middle plane showing highlighted segmented nucleus of the polymorphonuclear cell (arrow), due to accumulation of fluorescent drug-coupled nanoparticles; C, lower optical plane (at the level of cell contact with glass support).

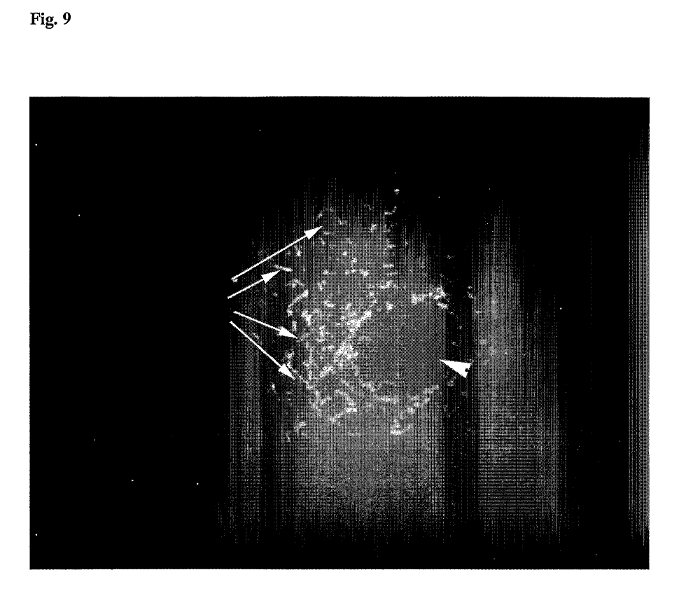

FIG. 9 illustrates the uptake of red CdTe nanoparticle in mitochondria in macrophages. Red CdTe nanoparticle were incubated with monocyte-derived macrophages for 15 mins in culture. Live cell confocal microscopy was then performed to examine red CdTe nanoparticle localisation.

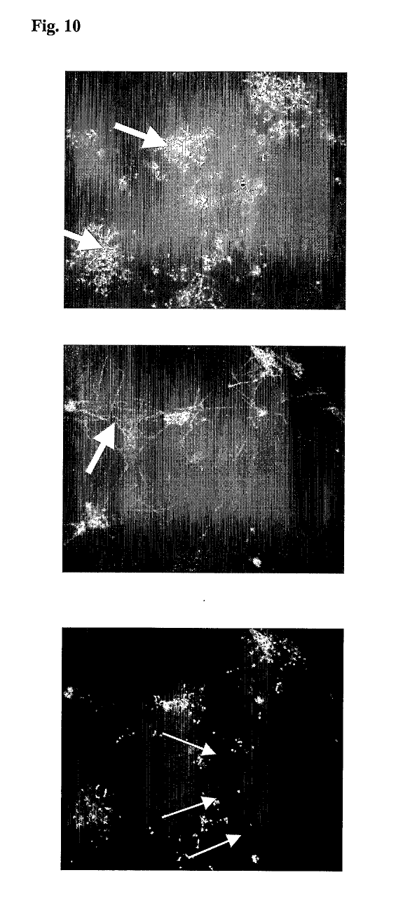

FIG. 10. illustrates the accumulation of red and green CdTe nanoparticles within the elements of the blood coagulation system. Fresh blood was allowed to clot for 15 mins at 37° C. in glass chambered slides. Immediately after clot formation the glass chambers were washed out with warm culture medium containing quantum dots and incubated at 37° C. for 15 minutes. The upper panel shows red CdTe nanoparticles highlighting groups of platelets while the 2.2 nm size green-emitting quantum dots decorate freshly formed fibrin filaments in the middle panel. The lower panel corresponds to the overlay of red green and blue channels, where by blue nuclei in the lower panel are dyed with Hoechst nuclear stain.

Referring to FIGS. 1 and 3, as an example without limitation to the present invention, a nanocarrier-based drug delivery system is used for treatment of an inflammatory condition accompanied by abnormally enhanced functional activity of phagocytic cells. Anti-inflammatory drugs are known to modulate macrophage function, but possessing non-specific undesirable side effects for the different cell types, are used in complex with medium-sized nanocarriers (3-8 nm diameter), which are subject to active engulfment and uptake by human macrophages. The system can be deployed at the site of inflammation by local application, e.g. direct injection into the inflamed joint. Overactive phagocytic cells at the site of inflammation will be exposed to enhanced drug uptake with subsequent moderation and/or resolution of inflammation.

Phagocytic cells have differential ability to uptake nanoparticles depending on their maturation and cell cycle status thereby increasing the opportunity of selective targeting of cell sub populations. The same principles can be applied to the cells of the non phagocytic lineages.

As other examples without limitation to the present invention, the nanodrug system is used for the treatment of inflammatory conditions accompanied by over-activity of polymorphonuclear phagocytes, protozoa-related infections, such as disenteria caused by amoebal parasites of the large intestine and infections caused by bacterial intestinal flora. The nanodrug system of the invention can be composed of the drug conjugated to the nanoparticles of the 5-10 nm size. Uptake of the drug by the cells, parasites and epithelial cells can be facilitated by active non-specific phagocytosis of the particles, at the same time creating the uptake barrier for the intestinal cells due to the carrier size. For examples of the drug-particle system within microphagocytes and epithelial cells refer to FIGS. 1-3.

In another preferred embodiment, referring to the FIG. 7, the nanosize drug delivery system is implemented for treatment of blood coagulation disorders associated with excessive clot formation and thrombosis. Fibrin-destabilising drugs coupled to nanoparticles can be delivered intravenously and due to the unique avidity of nanoparticle carriers to build into the biopolymers, the drug is selectively targeted to the intravascular sites of fibrin formation and exerts its fibrinolytic effects.

Another example of preferred embodiment of the invention without limitation to the one described here referring to FIG. 3 is the regulation of gene expression (gene therapy) using the nanosystems for targeted nuclear drug delivery. Drug-carrier complexes are applied to the living cells, selectively uptaken into the cytoplasm and subsequently into the nuclei on the basis of their size, charge and surface functionalisation specificity. The nanosystems are bound to the DNA or RNA in the nucleus and deliver the signal for the selectively targeted gene resulting in altered protein synthesis or changes in cell functional responses.

In another example of preferred embodiment referring to FIGS. 4 and 5, nanosystems are used for verification of intracellular drug transport and delivery efficiency. Fluorescent quantum dots can be coupled to the drugs under study and brought in contact with the living cells as described in the previous example. The efficiency of the drug delivery and specificity of intracellular distribution is subsequently evaluated by live cell confocal imaging.

In yet another preferred embodiment referring to FIG. 8, salicyl and aspirin-based drug systems can be constructed and delivered into cell.

As other example of preferred embodiment FIGS. 7 and 10 nanoparticle based drug delivery system may be used to target fibrin filaments. The nanodrug system of the invention can be composed of the drug conjugated to nanoparticles of 2-10 nm size, the drug thereby being incorporated into the fibrin clot during its development can selectively modify (enhance or reduce) the speed of its formation or composition.

Yet as other example of preferred embodiment FIG. 9, nanodrug delivery systems are used to target mitochondria. Selective targeting of such systems to mitochondria can be utilised for modifying cell functions e.g. targeting of both, small drug molecules and large macromolecules to and into mitochondria may provide the basis for a large variety of future cytoprotective and cytotoxic therapies: The delivery of therapeutic DNA and RNA such as antisense oligonucleotides, ribozymes, plasmid DNA expressing mitochondrial encoded genes as well as wild-type mtDNA may provide the basis for treatment of mitochondrial DNA diseases; the targeting of antioxidants into the mitochondrial matrix may protect mitochondria from oxidative stress caused by a variety of insults, perhaps even contribute to slowing down the natural aging process; the mitochondria-specific targeting of naturally occurring toxins or synthetic drugs such as photosensitizers may open up avenues for new anticancer therapies.

In another preferred embodiment delivering molecules known to trigger apoptosis by directly acting on mitochondria may overcome the apoptosis-resistance of many cancer cells and drugs able to target mitochondrial uncoupling proteins may become a basis for treating obesity.

In another example of preferred embodiment of nanodrug systems can be used to modify the conductivity or speed of electrochemical signal transduction mediators, (ions, synaptic vesicles, etc.) or polarisation events in nerve cells, cardiomyocytes, and other signal transducting cells. In this embodiment the semiconducting nature of the nanosystems can be exploited to alter and measure and manipulate the status of cell potential (relevant to the ratio between the cell membrane and cytosol). Consequently, a number of cell functions dependent on this parameter can be selectively targeted, inhibited or restored in case of a pre existing damage.

The nanosized drug delivery systems of the invention may include individual drugs or complex mixtures thereof. The compounds of therapeutic nature which have the potential and are anticipated to be used in such systems include but not limited to anti-inflammatory compounds such as aspirin, ibuprofen, and naproxen, mobic, Celebrex, disease-modifying anti-rheumatic drugs (DMARDs, methotrexate and sulphasalazine, anti-malarials (hydroxychloroquine), d-penicillamine, azathioprine and gold salts, transcription modulating drugs such as Thiazolidinediones, tamoxifen, anti cancer, anti bacterial drugs and antibiotics.

Salicyl and Aspirin-Based Nanodrug Systems

Structures of salycyl- and aspirin-functionalised nanoparticles are presented in Scheme 1. To prepare these nanoparticles an appropriate salycyl- or aspirin-containing precursor was synthesised first. 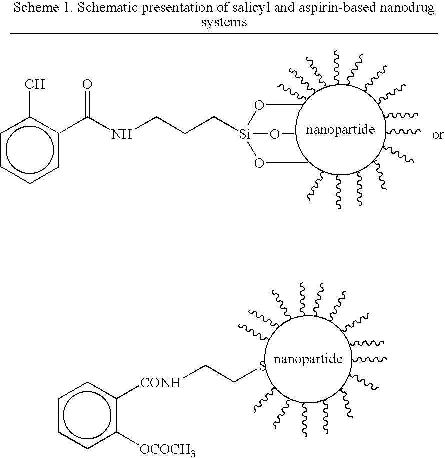
Preparation of Salicyl-Containing Precursors

The salicylamidopropyltriethoxysilane precursor was made by mixing 3-aminopropyltriethoxysilane with ethyl salicylate under argon and heating at ca. 120° C. for at least 24 hours to ensure complete migration of the salicylyl group from the ester to the amide (Scheme 2). This precursor was used for the preparation of the correspondent siloxane nanoparticles and functionalisation of CdTe and magnetite nanoparticles. In a similar manner, salycyl can be linked to cysteamine or to any appropriate compound containing amine functionality to give correspondent precursors for capping of nanoparticles. 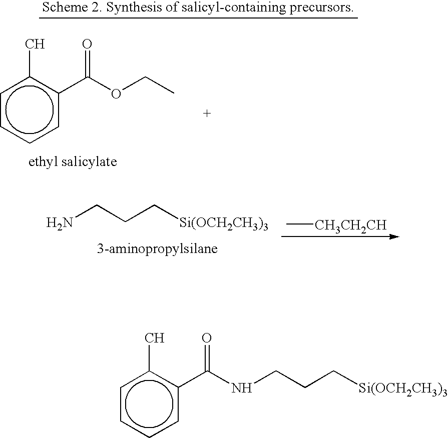
Preparation of Aspirin-Containing Precursors

The aspirin containing precursor was made by reaction of aspirin chloride with cysteamine (Scheme 3). In a similar manner aspirin can be linked to any appropriate compound containing amine functionality to give precursors for capping of nanoparticles. 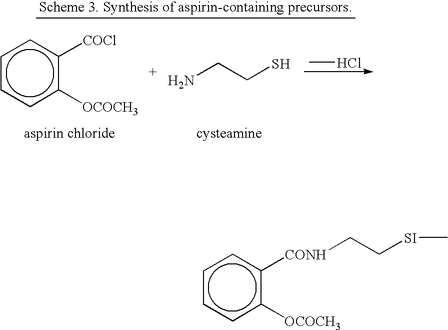
Preparation of Salicylyl- and Aspirin-Functionalised Siloxane Nanoparticles.

3-salicylamidopropyltriethoxysilane or a corresponding aspirin precursor was hydrolysed at room temperature in THF with H₂O. The white precipitate was dispersed in water to form a colloidal suspension, which is suitable for experiments performed in aqueous phase.

Preparation of Salycyl- and Aspirin-CdTe Nanocarrier Systems

Salycyl- and aspirin-conjugated CdTe nanodrug systems have been prepared similarly to the procedures above using an appropriate thiol precursor

Preparation of Salycy-l and Aspirin-Based Magnetite Nanodrug Systems.

Salycyl- and aspirin-functionalised magnetite nanoparticles have been prepared by the addition of 3-salicylamidotriethoxysilane or correspondent aspirin precursor to a suspension of Fe₃O₄ nanoparticles (size 9-11 nm) in THF. This was followed by the addition of degassed, deionised water, and the reaction mixture was left stirring vigorously at room temperature for 12 hours. The precipitate of functionalised magnetite nanocrystals was washed with THF and then dispersed in water using ultrasound. Then the samples were suitable for testing in cell culture systems.

Optical Characterisation

UV-vis absorption spectra of the colloidal solutions of nanocrystals were measured using a Shimadzu UV-3101 PC spectrometer and the photoluminescence (PL) spectra were recorded using a Spex Fluorolog spectrometer equipped with a R943 Hamamatsu photomultiplier. The optical density of all samples was kept the same and below 0.1 at the first absorption feature of the nanocrystals for a 1-cm path length.

Other non limiting examples of nanoparticles which can be used in relation to the invention may comprise semi conductor nanoparticles

II-VI semiconductor nanoparticles: ZnO, ZnS, ZnSe, ZnTe, CdS, CdSe, CdTe, HgS, HgSe, HgTe.

III-V semiconductor nanoparticles: AlN, AlP, AlAs, AlSb, GaN, GaP, GaAs, GaSb, InN, InP, InAs, InSb.

Group IV semiconductor nanoparticles: Si, Ge, Si₁₋ₓGeₓ

Other possible nanoparticles include SiO₂ (silica), any transition metal oxide (e.g. TiO₂, ZrO₂, HfO₂, MoO₂, Fe₂O₃, Fe₃O₄, CO₃O₄, ferrites), siloxane nanoparticles, dendrimers (dendritic polymers) and organic polymer nanoparticles.

Two aqueous colloidal solutions of CdTe nanocrystals of 2-5 nm mean size were used for studies in biological systems.

The entity to be delivered to a target site may be indirectly linked to a nanoparticle using an organic linker. Such a linker may be organic group, which can serve to link the stabiliser, drug or biomolecule to the nanoparticle surface such as: alkyl chain e.g.(—CH₂—)ₙ, polyethyleneglycole e.g.(—CH₂—O—CH₂—)ₙ, peptide e.g. (—CH₂)ₙ, —NH—CO—(CH₂—)ₙ), ester, disulfide.

The invention is not limited to the embodiments hereinbefore described which may be varied in detail.

### REFERENCES

Akerman M E, Chan W C, Laakkonen P, Bhatia S N, Ruoslahti E Nanocrystal targeting in vivo. Proc. Natl. Acad. Sci. USA. 2002, 99, 12617.
Bruchez M Jr, Moronne M, Gin P, Weiss S, Alivisatos AP Semiconductor nanocrystals as fluorescent biological labels. Science. 1998, 281, 2013.
Chan W C, Nie S. Quantum dot bioconjugates for ultrasensitive nonisotopic detection. Science. 1998, 281, 2016.
Chan W C, Maxwell D J, Gao X, Bailey R E, Han M, Nie S. Luminescent quantum dots for multiplexed biological detection and imaging. Curr. Opin. Biotechnol., 2002, 13, 40.
Dubertret B, Skourides P, Norris D J, Noireaux V, Brivanlou A H, Libchaber A In vivo imaging of quantum dots encapsulated in phospholipid micelles. Science. 2002, 298, 1759.
Gaponik N, Talapin D V, Rogach A L, Hoppe K, Shevchenko E V, Kornowski A, Eychmuller A, Weller H. Thiol-capping of CdTe nanocrystals: an alternative to organometallic synthesis routes. J Phys. Chem. B. 2002, 106, 7177.
Gaponik N, Radtchenko I L, Gerstenberger M R, Fedutik Y A, Sukhorukov G B, Rogach A L. Labeling of biocompatible polymer microcapsules with near-infrared emitting nanocrystals. Nano Lett. 2003, 3, 369.
Goldman E R, Anderson G P, Tran P T, Mattoussi H, Charles P T and Mauro J M. Conjugation of luminescent quantum dots with antibodies using an engineered adaptor protein to provide new reagents for fluoroimmunosays, Anal. Chem. 2002, 74, 841.
Jaiswal J K, Mattoussi H, Mauro J M, Simon S M. Long-term multiple color imaging of live cells using quantum dot bioconjugates. Nature Biotechnol. 2003, 21, 47.
Kim S, Lim Y T, Soltesz E G, De Grand A M, Lee J, Nakayama A, Parker J A, Mihaljevic T, Laurence R G, Dor D M, Cohn L H, Bawendi M G and Frangioni J V. Near-infrared fluorescent type II quantum dots for sentinel lymph node mapping, Nature Biotechnology, 2004, 22, 93.
Larson D R, Zipfel W R, Williams R M, Clark S W, Bruchez M P, Wise F W, Webb W W. Water-soluble quantum dots for multiphoton fluorescence imaging in vivo. Science. 2003, 300, 1434.
Mattoussi H, Mauro J M, Goldman E R, Anderson G P, Sundar V C, Mikulec F V and Bawendi M G. Self-assembly of CdSe—ZnS quantum dot bioconjugates using an engineered recombinant protein, J. Am. Chem. Soc. 2000, 122, 12142.
Mattoussi H, Huno M K, Goldman E R, George P, Mauro J M. Optical Biosensors: Present and Future. Elsevier, The Netherlands 2002. (eds. Ligler F S and Rowe C A) pp 537-569.
Michalet, X., Pinaud, F., Lacoste, T. D., Dahan, M., Bruchez, M. P., Alivisatos, A. P., Weiss. S. Properties of semiconductor nanocrystals and their application to biological labelling. Single Molecules. 2001, 2, 261.
Peng Z A, Peng X. Formation of high-quality CdTe, CdSe, and CdS nanocrystals using CdO as precursor. J. Am. Chem. Soc. 2001, 123, 183.
Seydel C. Quantum dots get wet. Science. 2003, 300, 80.
Wang S, Mamedova N, Kotov N and Chen W. Antigen/Antibody immunocomplex from CdTe nanoparticle bioconjugates, Nano Lett, 2002, 2, 817.

1-104. (canceled)

105: A method for targeting a specific site comprising the steps of:
evaluating characteristics of a target site; and
providing an entity dependent on the site characteristics, the entity comprising nanoparticles of up to 20 nm to target the entity to the specific site.

106: The method as claimed in claim 105 wherein the entity comprises nanoparticles of up to 10 nm.

107: The method as claimed in claim 105 wherein the entity comprises nanoparticles of up to 5 nm.

108: The method as claimed in claim 105 wherein the entity comprises nanoparticles of up to 3 nm.

109: The method as claimed in claim 105 wherein the nanoparticles are water soluble.

110: The method as claimed in claim 105 wherein the nanoparticles are lipid soluble.

111: The method as claimed in claim 105 wherein the nanoparticles comprises II-IV nanoparticles.

112: The method as claimed in claim 105 wherein the nanoparticles are CdTe nanoparticles.

113: The method as claimed in claim 105 wherein the nanoparticles are CdSe nanoparticles.

114: The method as claimed in claim 105 which comprises modifying the material composition, change, or any surface parameters of the entity.

115: The method as claimed in claim 105 wherein the target is selected from one or more of living cells, dead cells, a bacterium, a fungus, a eukaryotic life form, a prokaryotic life form, intracellular organelles.

116: The method as claimed in claim 105 wherein the target is within or on a biological membrane.

117: The method as claimed in claim 105 wherein the target site is extracellular to a biological membrane.

118: The method as claimed in claim 105 wherein the target is selected from one or more of mitochondria, endoplasmic reticulum, cells of the immune system, macrophages, the nucleus of macrophages, the cytosol of macrophages, a cellular or an acellular component of the blood coagulation system, platelets, neutrophils, fibrin.

119: The method as claimed in claim 105 wherein the entity is directly associated with a nanoparticle.

120: The method as claimed in 119 wherein the entity is directly linked to a nanoparticle.

121: The method as claimed in claim 119 wherein the entity is physically attached to a nanoparticle.

122: The method as claimed in claim 119 wherein the entity is chemically attached to a nanoparticle.

123: The method as claimed in claim 119 wherein the entity is conjugated to a nanoparticle.

124: The method as claimed in claim 105 wherein the entity is indirectly associated with a nanoparticle.

125: The method as claimed in claim 124 wherein the entity is indirectly linked to a nanoparticle using an organic linker.

126: The method as claimed in claim 105 wherein the entity comprises a stabiliser.

127: The method as claimed in claim 105 wherein the entity comprises a medicinal drug.

128: The method as claimed in claim 105 wherein the entity comprises a diagnostic or imaging agent or sensor.

129: The method as claimed in claim 105 wherein the entity comprises DNA, RNA, protein, or chemical, their derivatives, constituents thereof and modified products thereof.

130: A drug delivery system comprising a biologically active entity associated with a carrier comprising nanoparticles of up to 20 nm.

131: The system as claimed in claim 130 wherein the entity is directly associated with a nanoparticle.

132: The system as claimed in claim 130 wherein the entity is directly linked to a nanoparticle.

133: The system as claimed in claim 131 wherein the entity is physically attached to a nanoparticle.

134: The system as claimed in claim 131 wherein the entity is chemically attached to a nanoparticle.

135: The system as claimed in claim 131 wherein the entity is conjugated to a nanoparticle.

136: The system as claimed in claim 130 wherein the entity is indirectly associated with a nanoparticle.

137: The system as claimed in claim 136 wherein the entity is indirectly linked to a nanoparticle using an organic linker.

138: The system as claimed in claim 130 wherein the entity comprises a stabiliser.

139: The system as claimed in claim 130 wherein the carrier comprises nanoparticles of up to 10 nm.

140: The system as claimed in claim 130 wherein the carrier comprises nanoparticles of up to 5 nm.

141: The system as claimed in claim 130 wherein the carrier comprises nanoparticles of up to 3 nm.

142: The system as claimed in claim 130 wherein the nanoparticles are water-soluble.

143: The system as claimed in claim 130 wherein the nanoparticles are lipid soluble.

144: The system as claimed in claim 130 wherein the nanoparticles comprise water-soluble II-VI nanoparticles.

145: The system as claimed in claim 130 wherein the nanoparticles are CdTe nanoparticles.

146: The system as claimed in claim 130 wherein the nanoparticles are CdSe nanoparticles.

147: The system as claimed in claim 130 for use in targeting a biological object.

148: The system as claimed in claim 147 wherein the target is selected from one or more of living cells, dead cells, a bacterium, a fungus, a eukaryotic life form, a prokaryotic life form, intracellular organelles.

149: The system as claimed in claim 147 wherein the target is within or on a biological membrane.

150: The system as claimed in claim 147 wherein the target is extracellular to a biological membrane.

151: The system as claimed in claim 147 wherein the target is selected from one or more of mitochondria, endoplasmic reticulum, cells of the immune system, macrophages, the nucleus of macrophages, the nucleolus of phagocytes, the cytosol of macrophages, a cellular or an acellular component of the blood coagulation system, platelets, neutrophils, fibrin.

152: The system as claimed in claim 130 wherein the nanoparticles are II-VI nanoparticles.

153: The delivery system as claimed in claim 130 wherein the nanoparticles are organic or inorganic nanoparticles.

154: The system as claimed in claim 130 wherein the entity is selected from one or more entities which interact with and undergoes modification upon contact with cell membranes or their components, and in turn modifies their function, interacts with and undergoes modifications upon contact with cytoplasmic components, and in turn modify their function, interacts with and undergoes modifications upon contact with cytoskeletal components, and in turn modify their function, interacts with and undergoes modification upon contact with nuclear components, and in turn modify their function, interacts with and undergoes modifications upon interaction with extracellular matrix, and in turn modify their function, interacts with and undergoes modification with blood constituents, interacts with and undergoes modification upon interaction with cell membrane, interacts with and undergoes modifications upon interaction with membranes of subcellular organelles, interacts with and undergoes modifications upon interaction with nuclear membrane, interacts with and undergoes modification upon interaction with nuclear pores, is targeted to selected intracellular compartments by an external magnetic field, is targeted to selected intracellular compartments by external optical illumination, is targeted to selected intracellular compartments by modification of intracellular pH, is targeted to selected subcellular organelles by external magnetic field, is for treatment of cancer and other diseases accompanied by abnormal cell and tissue function, is for treatment of inflammatory conditions, is targeted to the intestinal epithelium, bacterial or parasitic flora utilised for treatment of infections of gastrointestinal tract, is for treatment of diseases and/or post traumatic conditions of the nervous system and/or nerve cells, the entity is for treatment of coagulation disorders and cardiovascular diseases.

155: A probe comprising nanoparticles of less than 5 nm in size.

156: A probe comprising nanoparticles of less than 3 nm in size.

157: The probe as claimed in claim 155 wherein the nanoparticles are of CdTe.
